(19) United States
(12) Patent Application Publication
(10) Pub. No.: US 2003/0228375 A1
(43) Pub. Date: Dec. 11, 2003

(54) Composition and method for increasing testosterone levels

(75) Inventor: Inna Yegorova, Northridge, CA (US)

Correspondence Address: SIERRA PATENT GROUP, LTD., P O BOX 6149, STATELINE, NV, 89449, US

(73) Assignee: A. Glenn Braswell

(21) Appl. No.: 10/133,767

(22) Filed: Apr. 25, 2002

(51) Int. Cl. A61K 35/78; A61K 33/32; A61K 31/57; A61K 31/19; A61K 31/015

(52) U.S. Cl. 424/643; 424/727; 424/758; 424/764; 424/767; 424/778; 514/170; 514/574; 514/765

(57) ABSTRACT

This invention provides compositions and methods related to the administration of deer antler, one or more nor-testosterone precursors, and one or more testosterone precursors, to increase testosterone levels, treat sexual dysfunction, improve sexual function, improve energy, enhance feelings of well-being and increase muscle mass in males. This invention also provides for inhibitors of the enzymes aromatase and/or 5-alpha reductase, to support testosterone levels and avoid undesirable metabolites.

### FIELD OF THE INVENTION

This invention relates generally to novel compositions and related methods that combine deer antler, testosterone, testosterone precursors and nor-testosterone precursors, and aromatase and 5-alpha reductase inhibitors in order to increase testosterone levels, treat sexual dysfunction, raise energy levels, improve sexual function, enhance feelings of well-being and increase muscle mass in the human male.

### BACKGROUND OF THE INVENTION

Testosterone is the primary androgen or male reproductive (sex) hormone produced naturally in the body. Normal male sexual development, including the sex organs, increases in muscle mass, facial hair, and deep voice, depends on testosterone. In adult males, testosterone effects maintenance of muscle and bone mass, sexual function and psychological well being. As males grow older, however, especially after the age of 35, testosterone levels decline slowly, accompanied by symptoms that have been associated with the condition known as “andropause.” Symptoms of andropause include lethargy, depression, lack of sexual desire and function, and loss of muscle mass and strength.

Men suffering testosterone deficiency have many replacement therapies available, but each has particular disadvantages. For example, injections of testosterone esters in oil depot form have been used for decades, but these injections are often both inconvenient and painful. Moreover, these injections result in inconsistent testosterone levels in the blood: a supraphysiological surge in testosterone level is seen soon after injection, but by the time of the next injection, testosterone levels have often dropped below standard physiological levels. These supraphysiological surges may increase the incidence of undesirable side effects (e.g.,. prostrate hypertrophy) as well as amplify the shutdown of the hypothalamic/pituitary testicular axis (HPTA). Goodman & Gilman Sec XIII-Hormones and Hormone Antagonist (9^{th} Ed. 1996). Testosterone is also available as a transdermal system, applied to the scrotal skin, but this causes a disproportionate increase in plasma dihydrotestosterone (DHT) levels due to conversion by the scrotal skin during absorption. Goodman & Gilman (1996).

Several testosterone precursors and derivatives, such as androstenedione (see U.S. Pat. No. 5,578,588), 4-androstenedione, 4-androstenediol (see U.S. Pat. No. 5,880,117), 5-androstenedione, 5-androstenediol and their nor-derivatives have been proposed for testosterone supplementation. Many of these are available commercially. The administration of these steroid precursors is not without risk, however, because substances that will enhance testosterone will also enhance the production of DHT, a metabolite that is the more active molecule in peripheral tissues such as the prostate and hair follicles. Moreover, testosterone and its androstenedione precursors are aromatized into estrone and estradiol, respectively, with known estrogenic effects including breast enlargement (gynecomastia).

Regarding nor-derivatives, these molecules have testosterone's anabolic effects of maintaining muscle and bone mass, without the unwanted androgenic effects such as aggravation of prostate and/or male pattern baldness problems. These include, for example, 17β-ester of nandrolone (See U.S. Pat. No. 4,083,973); 7α-methyl-19-nortestosterone (See U.S. Pat. No. 5,342,834); and 19-nor4-androstenediol, 19-nor-4androstenedione, 19-nor-5-androstendione, and 19-nor-5-androstenediol (See U.S. Pat. No. 6,011,027). Two particular embodiments of the nor-testosterones, norethandrolone and ethylestrenol, are alkylated molecules, providing greatly improved oral bioavailablity compared to the non-alkylated steroids. Alkylation, however, has been associated with a greatly increased risk of hepatotoxicity. Therefore, these synthetic compounds are far from an ideal solution

Another approach in increasing testosterone levels includes the use of steroid precursors and nutritional blends. For example, Acetabolan-II from Prime Nutrition (Fort Worth, Tex.) contains ingredients shown in scientific studies to support natural testosterone levels. Acetabolan-II may also significantly improve the affinity of androgen receptor sites, thus supporting the anabolic effects of natural testosterone. The main ingredients in Acetabolan-II are acetyl-L-carnitine, tribulus terrestris and “ZincTech” (chelated zinc, magnesium, and vitamin B6). Acetabolan-II is claimed to elicit a higher testerone to cortisol ratio, thereby supporting a strong anabolic environment that fosters maximum muscle growth and recovery. No human trials support this theory, however.

The invention herein provides a novel approach to testosterone therapy, combining synthetic testosterone precursors and nor-testosterone precursors with natural forms of these hormones provided in velvet deer antler. Velvet deer antler, used for twenty centuries as a powerful restorer, strengthening, healing and improving tissue function, has been shown recently to increase testosterone levels. Deer antler offers many of the benefits of the popular androgenic pro-hormones used today, and, when administered with the synthetic testosterone derivatives, modulates the effects of those pro-hormones. Hence, the present invention combines natural and synthetic hormone precursors that increase testosterone levels and improve sexual function, mood, physical endurance, strength, and lean muscle mass. The present invention also combines these testosterone forms with herbal and mineral ingredients that support testosterone levels and suppress unwanted side effects.

### SUMMARY OF THE INVENTION

The present invention relates to compositions and methods for increasing testosterone levels, treating sexual dysfunction, improving sexual function, improving energy, enhancing feelings of well-being and increasing muscle mass, by administering deer antler, a tonifying substance, in combination with testosterone and nor-testosterone precursors.

The compositions of the present invention preferably comprise deer antler, 19-nor-4-androstenedione, 19-nor-5-adrostenedione, 19-nor-5-androstendiol, 19-nor-4androtenediol, 4-androstendione, 5-androstendione, 4-androstenediol, and 5-androstenediol. In a specific embodiment of the present invention, the composition may comprise a pharmaceutically acceptable carrier.

In another embodiment of the present invention, the combination also comprises chrysin, and/or a substance that controls 5-alpha-reductase. That substance may be selected from the group consisting of Serenoa repens, cactus flower, Zinc, Azelaic acid, Dalbergia cochinchinensis, Sabal serrulata, Epilobium, Curcubitae pepo seeds, Urtica dioica root, and Polinis siccae extract.

A further embodiment of the present invention relates to a composition comprising deer antler in an amount between 5 mg and 300 mg, and between 5 mg and 300 mg each of 19-nor-4-androstenedione, 4-androstenedione, 19-nor-5-androstenedione, 5-androstenedione, 19-nor-5-androstenediol, 5-androstenediol, and chrysin.

In another embodiment of the present invention, the composition may be administered to a human male. The composition may also be administered orally, preferably two to three times daily.

### BRIEF DESCRIPTION OF THE DRAWINGS

FIG. 1 presents the mineral composition of the tip, upper, mid and base sections, and complete velvet deer antler. New Zealand Game Industry Board Draft Technical Manual, New Zealand Game Indus. Board (1998).

FIG. 2 illustrates the amino acid content of eight sections of velvet deer antler, number 1 representing the tip and number 8 representing the base sections, respectively.

FIG. 3 presents the collagen and fatty acid composition of the tip, upper, mid and base sections, and complete velvet deer antler harvested from Canadian wapiti. New Zealand Game Indus. Board (1998).

FIG. 4 shows the human testosterone pathway. KEGG Metabolic Pathways: Androgen & estrogen metabolism—Homo sapiens” (visited 2000) <http://www.genome.ad.jp/kegg/metabolism.html>.

### DETAILED DESCRIPTION OF THE INVENTION

The present invention relates to compositions, preferably dispersed in a pharmaceutically acceptable carrier, comprising testosterone and nor-testosterone precursors, preferably in combination with a tonifying substance to modulate the metabolism of these hormones. By using velvet deer antler along with the testosterone and nor-testosterone precursors, the antler promotes youthful testosterone levels while balancing and ameliorating dangerous spikes in these levels. Another embodiment of the invention includes herbs that inhibits 5-alpha-reductase reducing undesirable levels of dihyrotestosterone. Another embodiment includes chrysin, which inhibits aromatase and the production of estrogenic steroids.

Deer Antler

Deer antler (called Rokujo in Ancient Chinese Medicine) is used for its sexual-reinforcing and anti-aging actions. Wang et al., 36(7) Chem. Pharm. Bull. 2587-92 (1988). Velvet antler is living tissue that grows at a rate of up to 2 cm/day in some species. Cartilage, bone and support tissues such as nerves, blood vessels and hair follicles of the antler also evidence accelerated growth. Antler is the only mammalian organ that regenerates. These features, responsible for the accelerate growth of velvet antler are likely to be caused by either unique regulatory substances or substances found in other tissues but at lower levels. It is believed that factors actually responsible for the rapid regeneration of the velvet antler can explain the powerful health benefits of the product. Specifically, velvet deer antler regulates the adrenal cortex and energy metabolism, promotes sexual function and growth, and strengthens resistance. Its functions fall into the major categories of general body strengthening, healing, promoting blood cell growth and improving immune and cardiovascular function.

Some of velvet deer antler's key ingredients include lysophosphatidyl choline, with hypotensive activity, phosphatidyl ethanolamines, sphingomyelin, phosphatidyl choline hypothanthene and uridene, with monoamine oxidase (MAO)—inhibiting and anti-aging effects; polyamines spermine, spermidine and putrescine, with RNA polymerase stimulating effects; gangliosides that may promote memory and learning; and anti-inflammatory amino acids. A wide variety of growth factors are also found in velvet, and may be associated with its growth-promoting activity. Tsujibo et al., 35(2) Chem. Pharm. Bull. 654-59 (1987).

As taught by ancient Chinese medicine, deer antler tonifies the yang, primarily deficient yang of the kidneys, spleen and heart. Because kidneys are the seat of the basal yang, the most important use of this class of herbs is to tonify the kidney yang, whose principal manifestation of deficiency is systemic exhaustion. Yang deficiency causes impotence, spermatorrhea, watery vaginal discharge, infertility, enuresis, polyuria, wheezing and daybreak diarrhea. Patients with deficient kidney yang very often have decreased plasma thyroid hormone binding proteins, 24-hour urinary 17-ketosteroids, and decreased rate of glycolysis. When treated with tonifiers such as deer antler, these measurements return to normal ranges. Bensky et al., Chinese Herbal Medicine, Materia Medica, Revised Edition Eastland Press, Seattle, Wash. (1993).

Animal studies have elucidated the biochemical mechanism for some of deer antler's physiological effects. For example, an increase in testosterone levels has been shown in antler-fed mice. Specifically, senile-accelerated prone (SAM-P) mice appear senescent at one year of age, as compared to senile accelerated resistant mice (SAM-R). The plasma testosterone of SAM-P mice is half of that of the SAM-R strain. Repeated oral administration of Rokujo increased testosterone in both strains, but in SAM-P mice and not SAM-R, a dose dependent and statistically significant increase in plasma testosterone concentration was observed. Rokujo treatment also brought the decreased levels of the natural anti-oxidant super-oxide dismutase in SAM-P mice relative to SAM-R significantly towards normal (SAM-R) levels, and significantly inhibited MAO-B, known to increase with aging in both strains but more so in the SAM-P mice. Moreover, the incorporation of radio-labelled amino acid into RNA and DNA is increased by deer antler (Rokujo) extract both by in vivo administration to senile-accelerated mice and in vitro treatment of mouse liver. Wang et al., 36(7) Chem. Pharm. Bull. 2593-8 (1988).

Others have shown that pantocrine, an active ingredient of velvet deer antler, increased the weights of the prostate and seminal vesicles of young rats, but to a lesser degree than testosterone, as well as erythrocytes, hemoglobin, reticulocytes and leukocytes, and increased brain, liver, and kidney oxygen consumption in these rats. Chang et al., 2 Pharmacology & Applications Chinese Materis Medica, World Scientific (1986).

Researchers have also undertaken limited trials in humans, studying Rokujo's muscle-strengthening effects. Specifically, a New Zealand group conducted a double-blind trial of twenty-four healthy male volunteers, comparing effects of 70 mg antler velvet extract per day to placebo. The subjects trained their leg extensor muscles for three days a week and were tested twice pre training and twice after ten training weeks. Measurements included a resistance training apparatus for strength, a Biodex isokinetic dynamometer for endurance, and a wingate test for power. The increase in total work done (muscular endurance) by extension muscles of the Rokujo group was about twice that of placebo. Gerrard et.al., “Clinical Evaluation of New Zealand Deer Antler on Muscle Strength & Endurance in Healthy Male University Athletes,” Human Performance Centre, School of Physical Education Univ. Otago, AgResearch Ivermay, (unpublished data).

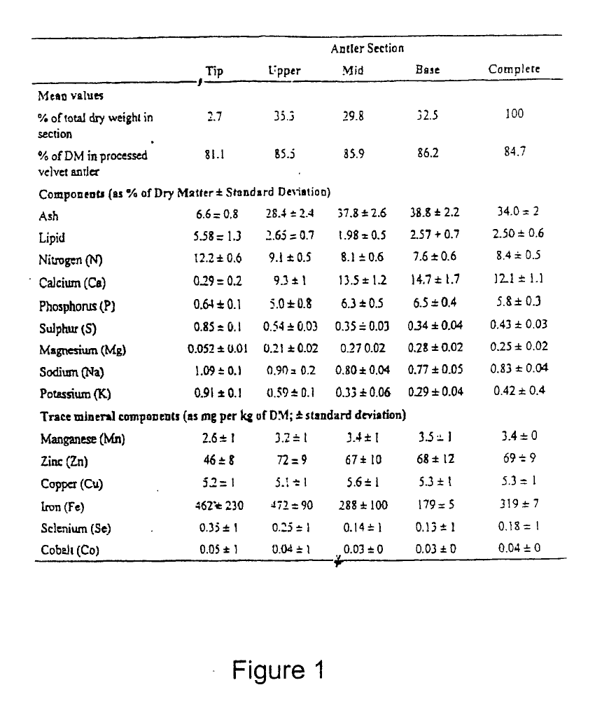

The mineral and lipid content of red deer velvet antler is shown in FIG. 1. New Zealand researchers processed the antlers from seventeen stags and analyzed them using standard laboratory procedures. For analysis, the antlers were separated into four major portions (tip, upper, middle and base). New Zealand Game Indus. Board (1998).

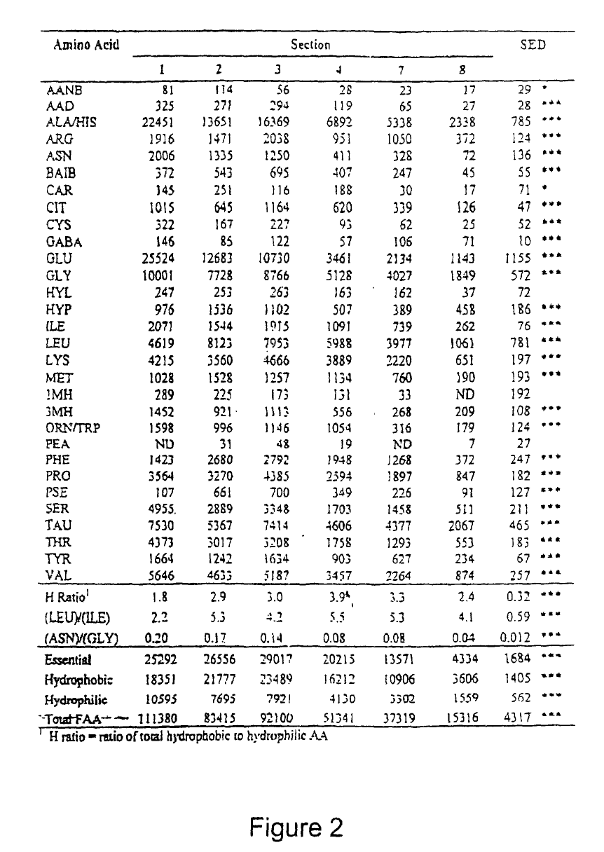

The free amino acid BAA) concentrations of velvet antler were measured in more detailed sections of the antler as shown in FIG. 2. The sections one through eight begin with one being at tip only 3 and 7 are in the tines and the remainder are in the main beam with eight being the most proximal. Levels of FAA are higher in the tip and upper sections with the highest levels in the tip itself, which is the zone of growth. Id.

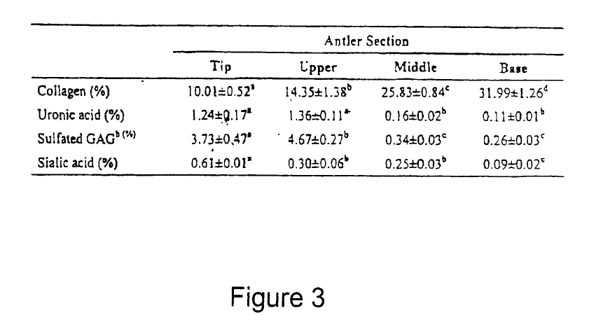

The collagen, sulfated glycosaminoglycans, uronic and sialic acid contents of Candanian Wapiti (elk) velvet antler are depicted in FIG. 3. The highest levels of these components are found in the tip and upper regions of the antler. New Zealand Game Indus. Board (1998) (quoting Sunwoo et al., 43 J. Ag. & Food Chem. 2846-49 (1995)).

Summarizing FIGS. 1, 2 and 3, it is clear that lipid and protein are more concentrated in the tip than in the base of the velvet antler. Conversely, ash and calcium remain more concentrated at the base. This reflects the fact that mineralization of the antler from the initial matrix of cartilage at the base of the antler and then extends to the tip. The active ingredients in velvet antler extracts are likely to be the proteins or lipids, which explains why the upper parts of the velvet are more heavily prized for their efficacy.

Velvet deer antler may be obtained from many species of deer, including New Zealand Red Deer and Canadian Wapati (Elk). Velvet Deer Antler is available from Ag Research, a company owned by the state of New Zealand that raises stags in a clean natural environment. Antlers are harvested using a humane process that causes no stress or injury to the animals and has the approval of veterinarians agencies and animal welfarists. Detailed analysis of its composition of ash, lipid, nitrogen, calcium, phosphorus, sulfur, magnesium, sodium, potassium, trace minerals, amino acids, fatty acids, collagen, glycoaminoglycans and fatty acids are depicted on FIGS. 1 through 3. New Zealand Game Induatry board Draft Technical Manual, New Zealand Game Industry Board, (1998).

Velvet deer antler may be obtained from other sources, and these preparations should have similar qualities and components as described in the New Zealand Game Industry Board Draft Technical Manual. Any equivalent deer antler could be used, such as the varieties available in China or Russia, that have also been shown to have restorative and sexual enhancing effects. Other sources include Gold Mountain Trading Co. (New Zealand), Coastal Nutrition Laboratories, Inc. (W. Hollywood Calif.), Tea Garden (W Hollywood Calif.), and BioSynergy Nutriceuticals (Sausalito, Calif.).

The velvet antler is made from all of the antler including bone and cartilage. The antler is harvested from the stag about half-way through the growth process, 50-60 days after growth begins, and frozen within three hours, then processed to remove the water content. The antler is renewable and can be removed each year without harming the animal. In traditional Chinese medicine, the velvet was dipped into near boiling water to cook then dried in the oven, followed by cool air drying. In New Zealand, steam replaces hot water dripping, and recently freeze drying has been used to preserve velvet without heat. A processed antler is typically 30-35% of its pre drying weight.

Velvet antler may be processed further into liquid form. In one method, it is soaked in alcohol and finely sliced, then the slices can be made into a soup with or without other herbs. In another method, it can be finely ground into a powder then encapsulated or made into an extract using either water or alcohol that can either be used as liquid, evaporated to give antler grease, or freeze dried. Powders can be encapsulated or added to other ingredients.

By these methods, the following estimated yields are obtained from 1 kg of green antler (a typical red deer produces 3-4 kg of antler):

| | | |
|---|---|---|
| Processed velvet: | 330 | gm |
| Dried powder: | 300 | gm |
| Freeze dried aqueos extract: | 45 | gm |
| Alcohol extract | 7.5 | gm |

In China and Korea, the practice combines the velvet antler extract in combination with other herbs to amplify positive effects for specific functions. Recommended doses in China are 900 mg to 1200 mg/day of the powder-in-liquor form, and 300 mg to 400 mg/day of powder boiled in water. In Russia, dose levels of prescriptions of 25 drops to 40 drops are calculated as equivalent to 750 mg to 1.2 g of ground dried velvet powder. In New Zealand, doses of 250 mg to 1200 mg per day are used. AgResearch. Velvet antlers yields gradual improvements in many tissues, it may take some time before the individual notices its benefits.

Testosterone and 19-nor-testosterone Precursors

Testosterone, 19-nortestosterone and its derivatives have been shown to increase blood testosterone levels, treat sexual dysfunction, improve sexual function and improve feelings of well being. A significant decrease in free testosterone, androstenedione, 5-androstenediol accompanies aging. Testosterone levels decline slowly and continuously throughout adult life in men, but the levels of dihydrotestosterone do not decrease with age. (Partin et.al., 145 J. Urology 405-9 (1991). Benign hyperplasia tissue in the prostate also increases with age, and has been correlated with circulating levels of free testosterone, estriol and estradiol, but not dihydrotestosterone.

Some methods of treating testosterone deficiency have been discussed above. Another method of increasing testosterone levels is the ingestion of the 17-ester form of testerone. Long-acting parenteral testosterone esters are used principally for long-term replacement therapy in men with androgen deficiency. Some esters, such as testosterone enanthate and testosterone cypionate, are long-acting and available as single-component injections. Testosterone propionate is shorter-acting, but allows for a more rapid onset of action if combined with the longer-acting esters. The esters are slowly absorbed from intramuscular injection sites. All preparations provide sustained testosterone activity for at least two weeks. Metabolic pathways of testosterone and its derivatives are similar to those of testosterone. Finally, testerone undecaoate is available by prescription and its side effects are similar to those observed with testosterone.

The testosterone precursors are normally metabolized from dehydro-epiandrosterone (DHEA). This has been studied in people with panhypopituitarism (lack of adrenal and gonadal steroids) by administering 50 mg or 200 mg of DHEA. This induces an increase of both steroids to supraphysiological plasma levels and a small increase of delta 5-androstenediol. In contrast, the increase of plasma delta 4-androstenedione was significant and dose dependent. DHEA was also converted into testosterone. The administration of a 50 mg dose of DHEA restored plasma testosterone to levels similar to those observed in young women. The 200 mg dose induced an important increase of plasma testosterone, slightly below the levels observed in normal men. The increase of plasma dihydrotestosterone levels was small at both doses of DHEA, in contrast with the large conversion of DHEA into androsterone glucuronide and androstanediol glucuronide. Finally, DHEA administration induced a significant and dose dependent increase of plasma estrogens and particularly of estradiol. Young et al., 82(8) J. Clin. Endocrinol. & Metabol. 2578-85 (1997).

Like testosterone, the administration of 19-nor-testosterone (nandrolone) exerts an anabolic effect that would be expected to increase muscle mass. Studies performed using injectable 17 beta-esters, such as nandrolone phenylpropionate, nandrolone decanoate and methenolone oenanthate exert a strong anabolic action for several weeks, amounting to 2.0 g to 2.50 g nitrogen/day, which corresponds to a daily gain of 12 g to 15 g protein or 60 g to 75 g lean body mass. Van Wayjen 143(14-15) Wiener Medizinischr Wochenschrift 365-75 (1993).

In another, double blinded, study, thirty healthy young men received testosterone enanthate (TE) or 19-nor-testosterone decanoate (ND), at 100 mg/wk or 300 mg/wk for six weeks. Of fifteen circumferences, significant increases were observed only for men receiving TE-300 mg/wk (shoulders) and ND-300 mg/wk (shoulders and chest). Friedl et al., 40(4-6) J. Steroid Biochem. & Mol. Biol. 607-12 (1990). Superior increases in the lean body mass of body builders ingesting nandrolone have been observed by other groups. Kuipers et al., 12(4) Int'l J. Sports Med. 413-18 (1991). These findings, however, have not been consistent among researchers. (Kuipers et al., 54(2) J. Appl. Physiol. Resp. Environ. & Exer. Physiol. 366-70 (1991). This steroid, however, induced a 25-27% decrease in HDL-cholesterol and diastolic blood pressure, both of which have well known cardiovascular risks. Kuipers (1991). Other groups have noted azoospermia (lack of sperm) associated with nandrolone use. Schurmeyer et al., 1(8374) Lancet 417-20 (1984).

The synthetic steroid 7α-methyl-19-nor-testosterone (MENT), a substituted 19-nortestosterone, is a potent androgen that is resistant to 5alpha-reductase. See, U.S. Pat. No. 5,342,834. It is not alpha-reduced because of steric hindrance and has been shown be four- to five-times more androgenic than testosterone, as measured by prostate and seminal vesicle weights. Moreover, MENT is ten-times as potent as testosterone in anabolic effects measured in the levator ani muscles. The nor-androgens as a group are more anabolic than androgenic. Sundaram et al., 53(1-6) J. Steriod Biochem. 253-57 (1995). MENT, however, while resistant to 5-alpha-reduction, is aromatized to form estrogenic compounds. Sundaram et al., 49 Recent Progress in Hormone Research 373-376, Academic Press (1994).

Researchers have also compared the effects of MENT and testosterone enanthate (TE) on sexual interest and activity, spontaneous erection and mood states, in twenty Caucasian and Asian hypogonadal men. In the Caucasian group, both MENT and TE treatments resulted in significant increases in sexual interest and activity, spontaneous erection (both by self-report and nocturnal penile tumescence (NPT) measurement), and increases in positive moods with decreases in negative moods. In the Asian group, both treatments increased waking erection, with a trend toward increased sexual interest and activity. These results demonstrate that MENT has effects similar to those of testosterone on sexual activity and mood states in hypogonadal men. As NPT is a physiological androgen-dependant outcome, these data provide further evidence for MENT's androgenicity. Anderson, 84(10) J. Clin. Endocrinol. & Metabol. 3556-62 (1999).

4-androstenedione and 4-androstenediol

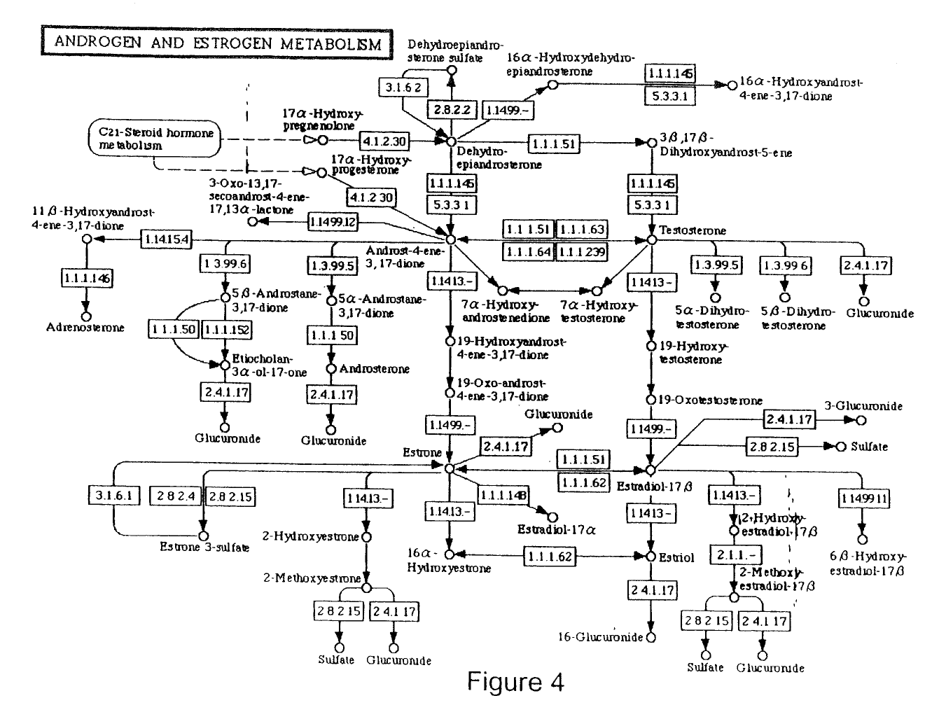

The androgens 4-androstenediol and 4-androstenedione are natural testosterone precursors. The biosynthesis of testosterone takes place within the testicular Leydig cells in two metabolic pathways. During the progesterone-pathway (δ-4 pathway), pregnenolone is metabolized to progesterone by the 3-β-hydroxy-steroid deydrogenase and an isomerase. Progesterone is then changed to 17-α-hydroxyprogesterone by the 17-α-hydroxylase and C₁₇C₂₁-lyase to androstenedione, then to testosterone by reduction of the 17-keto-group by 17-β-hydroxy-steroid dehydrogenase. The DHEA-pathway (δ-5 pathway) leads from pregenolone to 17-α-hydroxypregnenolone to dehyroepiandrosterone (C₁₇C₂₁-lyase), to 5-δ-androstenediol. See FIG. 4; Wichmann et al., 83(3) Exp. Clin. Endocrinol. 283-290 (1984).

As a testosterone pro-hormone and metabolite, 4-androstenedione may be used by athletes and bodybuilders to improve muscle mass. Levels of δ4-androstenedione increase significantly with moderate exercise in healthy men. Velardo et al., 97(l) Exp. & CLIN. Endocrinol. 99-101 (1991). Additionally, supplementation with 4-androstenedione has been known to produced elevations in serum testosterone. Mahesh et al., 41(3-8) J. Steroid Biochem. Mol. Biol. 495-513 (1992).

4-androstenediol is also metabolized into testosterone and is produced by conversion of dehyroepiandrosterone. Inaba et al., 13(2) Endocrinologia Japonica 160-72 (1966). It was first shown to produce elevations in human serum testosterone levels in 1965, and this was also demonstrated in in vitro studies in animals (Kundu, 6(5) Steroids 543-51 (1965) and human fibroblast cultures. Faredin et al. 32(2) Acta Medica Academlae Scientiarum Hungaricae 139-52 (1975). Supplementation with 4-androstenedione, 4-androstenediol, and 19-nor-4-androstenedione has been studied to determine whether a rise in testosterone is produced. Uralets et al., 23 Anal. Toxicol. 357-366 (1999). Testosterone is excreted in the urine unchanged and is metablized through 5α- and β-DHT as 5α- and β-androstanediol, while androstenedione is similarly excreted as androsterone and etiocholanolone. Both the final excreted steroid and the intermediaries stanediones and DHT intraconvert so that androsterone, 5α- and β-androstanediol and etiocholanolone are seen in urine. Supplementation with 4-androstenediol produced a 10-fold greater urine testosterone concentration than 4-androstenedione. (Uralets, 1999)

5-androtenediol and 5-androstenedione

The steroids 5-androstenedione and 5-androstenediol are secreted by the adrenal gland and in the testes, and are metabolites as well as precursors to testosterone. See Munabi et al., 63(4) So. J. Clin. Endocrinol. & Metabol 1936-40 (1986); Moger, 80(3) J. Endocrinol. 321-32 (1979). 5-androstenediol is a natural hormone with androgenic activity. Chang et al. 96(20) Proc. Nat'l Acad. Sci. 1173-77 (1999); Rosner et al.,15(1) Steroids 181-93(1970). In vitro studies reveal that 5-androstenediol is a precursor of both androstenedione and testosterone. Sulcova et al., 70(1) Endokrinologie 6-12 (1977).

The 4- and 5-androstenediols are part of two different pathways that predominate differently in different mammalian species. Precursors of the δ5-pathway (DHEA, androstendiol) are low in the red deer, dog, cat, rat and guinea pig. In comparison, precursors of the δ4-pathway (progesterone, 17-hydroxprogesterone, androstendione) are lower in the bull, boar, rain, stallion and rabbit. Wichmann et al., 83(3) Clin. Endocrinol. 283-90 (1984), 5-androstenediol is reported to have minimal androgenic activity and the potential to bind to estrogen receptors in several systems in women. Bird et al., 99 Acta Endocriniolgica 309-13 (1982). FIG. 4 depicts the pathway of humans.

Conversion of 5-androstenediol to radiochemically pure testosterone was demonstrated in the pituitary, some brain structures, and ventral prostate of adult castrated male rats. Formation of dihydrotestosterone and dehydroepiandrosterone was also detected in these animals. The may in part explain the behavioral and brain virilization effects of 5-androstenediol. Perez et al., 29(5) Steroids 627-33 (1977). 5androstenediol has been considered to be important in the human male as an intermediate in the biosynthesis of testosterone by testicular tissue. In women as well, it is metabolised into dehyro-epiandrosterone (DHEA), 4-androstenedione, and testosterone in both post-menopausal and young women. Bird et al. (1982).

19-nor-derivatives

The nor-derivatives, without a carbon in the 19 position, are not metabolized back to the 19 carbon form. For this reason, a nor-derivative of the precursors is metabolized not into testosterone itself but into nor-testosterone. Nor-testosterone is commercially available by prescription as nandrolone, an anabolic steroid (Deca-Durabolin™, Organon, Inc., New Jersey). Sattler et al., 84(4) J. Clin. Endocrinol. & Metabol. 1268-76 (1999). In a study in HIV-infected men, significant gains in total weight, lean body mass, body cell mass, muscle size and strength were observed with pharmacological doses of nandrolone decanoate, and the increases in lean body mass and muscular strength were significantly augmented with progressive resistance training. Similar results were obtained in a placebo controlled study in patients on dialysis where lean body mass increased significantly in patients given nandrolone compared with patients given placebo. Johansen et aL 281(14) JAMA 1275-81 (1999).

The 19-nor-androgens follow a metabolic pathway similar to that of the endogenous androgens. 19-norandrostenedione is excreted mainly as nor-androsterone and nor-etiocholanolone, the same excretion products observed for nor-testosterone (Nandrolone). 19-nor-androstendione converts into nor-testosterone in the body. (Uralets, 1999). Its impact, however, is immediate and short since it is inactivated for the most part in first-pass (through the liver) metabolism before it reaches the body.

Body builders have used testosterone and nor-testosterone precursors now for many years. Their effects on protein deposition and energy levels were demonstrated in a study of constant infusion with mini-osmotic pumps of several steroid hormones in young female rats. Testosterone and 5-androstenediol increased the proportion of protein in the body compositition of female rats, but did not have a significant effect on lipid deposition or heat production. Nor-testosterone increased energy expenditure, fuelled in part by a higher food ingestion, a trait shared by 4-androstenedione, but not by the other androgens. The effect of androgens on body weight may thus be a combination of their actions on food intake, efficiency of protein deposition, and activation of heat production or lipid (energy) storage. Almost all of the hormones increased the efficiency of protein deposition. Nor-testosterone increased heat production and androstenedione increased lipid storage, but these results were not statistically significant. Lobo et al., 29(2) Biochem. & Mol. Biol. Int'l 349-58 (1993).

The administration of testosterone precursors as 19-nor derivatives offers an advantage in that when 19-nor-testosterone is 5α-reduced, its affinity for peripheral receptors and potency decreases in target tissues such as hair follicles and the prostate, while its anabolic effects on muscles are maintained. In the seminal vesicle, testosterone is converted to DHT, an thus increasing by seven- to eight-fold of its affinity to the androgen receptor. Nor-testosterone is also converted effectively by 5-α-reductase by this metabolism, resulting in a three-fold decrease in its affinity. Toth et al., 87(2) Exp. Clin. Endocrinol. 125-32 (1986); Bergink, 22(6) J. Steroid Biochem. 831-36 (1985).

Over the past few years, use of testosterone precursors and their derivatives has become popular amongst life-extensionists as well as athletes. Testosterone and 19-nor-testosterone precursors are available in bulk from companies such as Eiselt Research (Sweden). Additionally, 19-nor-androstenedione is available from Extreme Sports Nutrition, as Androstack 6 by Powerstar Products, as 4-androstenediol by Osmo (San Antonio, Tex.), and in other formulations available from Active Life, Inc. (Plancentia, Calif.), Prolab, Inc. (Tacoma, Wash.), and Medlean Products (Muscatine, Iowa).

Chrysin

Although increased testosterone levels show positive effects on sexual function, mood, and muscle mass, increased testosterone levels also produce increased levels of estrogen and dihydrotestosterone. These can produce feminizing effects, benign prostatic hypertrophy (BPH) and hasten male pattern baldness (MPB). When BPH volume and hormone levels were corrected for age, BPH volume correlated positively with free testosterone, estradiol, and estriol. Partin et al., 145(2) J. Urol. 405-09 (1991). Similarly, estrogenic hormones have undesirable effects on the human male, which may be lessened by combining an aromatase inhibitor with supplements of testosterone pro-hormones. Aromatase enzyme plays a crucial role in the production of estrone from testosterone and estradiol from androstenedione. Goodman & Gilman (1996). The pro-hormones, or precursors, are also precursors to estradiol via metabolism by aromatase.

Chrysin controls aromatase activity, and thus the production of estradiol and estrone, and provides an alternative embodiment of this invention. This embodiment may further comprise a substance that controls 5-alpha-reductase and its production of DHT.

Other aromatase inhibitors include substituted androstenediones. There is also evidence that aromatase is involved in the production of DHT, which is well known for its negative effects on the prostate and male pattern baldness. An in vitro rat testis cell suspension model was used to investigate the metabolism of tritiated testosterone, dihydrotestosterone, and androstenedione. In the presence of aromatase inhibitors and androstenedione, the metabolism was shifted towards 17-keto forms. This suggests that androstenedione and the derived aromatase inhibitors activate the 17β-hydroxysteroid-dehydrogenase in a product-activating manner. Thus, aromatase inhibitors may regulate the intratissular levels, not only of estrogens, but also of other hormonally active steroids like DHT and 5-androstenedione. Schroder et al., 31(4B) J. Steroid Biochem. 685-90 (1988).

Because of the usefulness of inhibiting aromatase in breast cancer patients, several synthetic aromatase inhibitors have been developed. See, e.g., U.S. Pat. No. 4,954,446. There are natural substances, however, such as chrysin, that have similar activity. Chrysin is a bioflavonoid found in propolis (bee pollen) and honey that has been demonstrated to be as potent and effective in inhibiting aromatase as the popular pharmaceutical, aminoglutethimide (AG). In aromatase enzyme assays, chrysin, 7,8 benzo-flavone (ANG), AG, flavone and genistein 4′-methyl ether (5,7-dihydroxy-4′-methoxyisoflavone, Biochanin A) were shown to inhibit aromatase. Chrysin and AG inhibited the enzyme by 50% at a concentration of 4.6 μM and 7.4 μM, respectfully, and only ANG had a high I₅₀ of 0.5 μM. Both Flavone and Biochanin A inhibited aromatase, but to a lesser degree. Campbell et al., 46(3) J. Steroid Biochem. Mol. Bio. 381-88 (1993). In screening for potential chemopreventives against cancer, chrysin was one of the three of flavonoids with the greatest aromatase-inhibiting activity, with an inhibitory concentration (IC) of 1.1 μg/mL. Jeong et al., 22(3) Archives Pharma. Res. 309-12 (1999).

Chrysin is available commercially from suppliers well known to those skilled in the art. For instance, chrysin may be obtained from Mass Quantities, Inc. (New York, N.Y.) and Netrition, Inc. (NY).

5-alpha-reductase Inhibitors

As noted above, testosterone supplementation may be linked benign prostatic hypertrophy (BPH) because it elevates levels of free testosterone, estradiol, and estriol. Partin, (1991). Moreover, in 64 men with prostate cancer, ages 42 to 71 years old, it was shown that with age there was a significant increase in the volume of BPH, a significant decrease in the serum levels of free testosterone, androstenedione, dehydroepiandronsterone (DHA), dehydroepi-androsterone sulphate (DHA-S), delta 5-androstenediol, and 17-hydroxypregnenolone, and a significant increase in sex hormone-binding globulin (SHBG), luteinizing hormone (LH), and follicle stimulating hormone (FSH). When BPH volume and hormone levels were corrected for age, BPH volume correlated positively with free testosterone, estradiol, and estriol. These data indicate that, with age, patients with larger volumes of BPH have higher serum androgen and estrogen levels, suggesting that serum androgen and estrogen levels may be factors in the persistent stimulation of BPH with age. Therapeutic attempts at lowering plasma testosterone levels, reducing estrogen levels, or blocking androgenic stimulation through other mechanisms may interfere with the progression of BPH with age. Partin (1991.)

In its, peripheral target structures, testosterone must be converted into 5α-androstan-17β-ol-3-one (androstanolone or dihydrotestosterone, or CHT), 5α-androstane-3α, 47β-diol (3alpha-diol) and 5α-androstane-3β, 17β-diol (3β-diol) to become fully active. Massa et al., J. Steroid Biochem. (1975) 6: 567-571. The metabolism of testosterone to DHT is catalyzed by 5-alpha-reductase, an enzyme found in the hypothalamas. In target tissues, such as the prostate and hair follicles, the active medabolite is the 5-α-reduced testosterone, DHT. This compound differs from testosterone only in that the double bond between carbons 4 an 5 is hydrogenated into a single bond. For this reason, several products are available by prescription that inhibit the 5-alpha reductase and the formation of DHT. See Wilson et al., 6 Prostate Supplement 88-92 (1966); U.S. Pat. Nos. 5,998,427; 5,372,996; and 5,017.568. Finasterine (Proscar ™), the most popular alpha reductase blocker is a 4-aza steroid that selectively and competitively inhibits the activity of 5-alpha-reductase.

Other 5-alpha-reductase inhibitors include Serenoa repens, cactus flower, zinc, azelaic acid, Dalbergia cochinchinensis, Sabal serrulata, Epilobium, Curcurbitae pepo seeds, Urtica dioica root, and Pollinis siccae.

Thus, in one embodiment of the invention, the composition additionally comprises Serenoa repens, one of the most widely studied 5-alpha-reductase inhibitors, used in Europe as a medical treatment of BPH. See e.g., Swoboda et al., 149(8-10) Wiener Medizinische Wochenschrift 235 (1999); Di Silverio et al., 37(2) Prostate 77-83 (1998); Plosker et al., 9(5) Drugs & Aging 379-95 (1996); Carraro et al., 29(4) Prostate :231-40, at 241-2 (1996).

In another aspect of the invention, the composition further comprises Sabal serrulata. Weisser et al. (1997); Toth, 28(3) Urol. & Nephrol. 337-48 (1996); Weisser et al., 28(5) Prostate 300-06 (1997); Vahlensieck et al., 111(18) Fortschritte der Medizin 323-6 (1993).

Other 5-alpha reductase inhibitors may be selected from the group consisting of zinc and azelaic acid (Stamatiadis et al., 119(5) Brit. J. Dermatol. 627-32 (1988); cactus flower (Jonas et al., 26(4) Urol. Res. 265-70 (1998); Dalbergia cochinchinensis (Pathak et al., 46(7) Phytochemistry 1219-23 (1997); Epilobium species (Ducrey et al., 63(2) Planta Medica 111-14 (1997); Onagraceae (Lesuisse et al., 59(5) J. Nat. Prod. 490-92 (1997); Curcurbitae pepo seeds (Vahlensieck et al., 114(31) Fortschritte Der Medizin 407-11 (1996); Urtica dioica root (Vahlensieck et al., 113(3) Fortschritte Der Medizin 37-40 (1995); and Pollinis siccae extract (Vahlensieck et al. (1996)).

Serenoa repens, cactus flower, zinc, azelaic acid, Dalbergia cochinchinensis, Sabal serrulata, Epilobium, Curcurbitae pepo seeds, Urtica dioica root and Pollinis siccae are available commercially, in bulk and wholesale, from suppliers well known to those skilled in the art. For instance, Curcurbitae pepo, Pollinis siccae, and Sabal serrulata are approved in Germany as treatments for prostatic hyperplasia and available from suppliers well known to those skilled in the art, such as Kürbissamnen (Germany).

In one embodiment of the invention, the composition comprises deer antler, 4-androstenedione, 19-nor-4-androstenedione, 5-androstenedione, 19-nor-5-androstendione, 5-androstenediol, and 19-nor-5-androstendiol.

Another embodiment of the invention relates to a method for increasing testosterone levels, improving sexual function, improving mood, enhancing feelings of well being and increasing muscle mass comprising administering to a human a composition comprising deer antler, 4-androstenedione, 19-nor-4-androstenedione, 5-androstenedione, 19-nor-5-androstendione, 5-androstenediol, and 19-nor-5-androstendiol and chrysin.

Any dosage form may be employed for providing the patient with an effective dosage of the composition. Dosage forms include solid and liquid preparations including tablets, capsules, dispersions, suspensions, solutions, capsules, transdermal patches etc. Tablets and capsules represent the most advantageous oral dosage unit form. Any method known to those of ordinary skill in the art may be used to prepare capsules, tablets, or other dosage formulations. Pharmaceutically acceptable carriers include binding agents such as pregelatinized maize starch, polyvinylpryrrolidone or hydroxypropyl methycellulose; binders or fillers such as lactose, pentosan, microcrystalline cellulose or calcium hydrogen phosphate; lubricants such as magnesium stearate, talc or silica; disintegrants such as potato starch or sodium starch; or wetting agents such as sodium lauryl sulfate. Tablets or capsules can be coated by methods well known to those of ordinary skill in the art.

According to one aspect of the invention, a composition is provided comprising a pharmaceutically acceptable combination of the composition and at least one carrier. Pharmaceutically acceptable carriers for inclusion into the present compositions include carriers most suitable for combination with lipid-based drugs such as diluents, excipients and the like which enhance its oral administration. Suitable such carriers include, but are not limited to, sugars, starches, cellulose and derivatives thereof, disintegrants, dispersants, wetting agents such as sodium lauryl sulfate, lubricants, stabilizers, tabletting agents, anti-oxidants, preservatives, coloring agents and flavoring agents. Reference may be made to Remington's Pharmaceutical Sciences, 17th Ed., Mack Publishing Company, Easton, Pa., 1985, for other carriers that would be suitable for combination with the present oxysterol(s) to render an orally ingestible composition. As will be appreciated, the pharmaceutical carriers used to prepare compositions in accordance with the present invention will depend on the administrable form to be used.

According to another embodiment of the invention, the present composition is formulated for oral administration. Solid or liquid oral dosage forms formulated in accordance with standard pharmaceutical practice may be employed. Capsules are a particularly useful vehicle for administering the present composition. Deer antler may be given in unit doses between 5 mg and 1 gm, preferably between 5 mg and 300 mg. Testosterone precursors, the 19-nor-testosterone precursors, chrysin and the 5-alpha-reductase inhibitors can be given in doses between 5 mg and 1 gm, preferably between 5 mg and 300 mg. The composition may be administered orally, or by other administration routes including suppository, spray, powder, liposome, dermal patch, inhalant, topical cream, lotion or ointment.

The administration of the composition is preferably in accordance with a predetermined regimen, which may be at least once daily and over an extended period of time for chronic treatment, and could last for one year or more, including the life of the subject. The dosage administered will depend upon the frequency of the administration, the blood level desired, other concurrent therapeutic treatments, the severity of the condition, whether the treatment is for improving sexual function or mood, the age of the patient, the degree of increase in testosterone desired, and the like.

The invention will be further illustrated by the following non-limiting examples:

### EXAMPLE1

A composition of the following formulation was prepared in capsule form by standard methods, as described above:

| | |
|---|---|
| Deer antler | 100 mg |
| 4-androstenedione | 10 mg |
| 19-nor-4-androstenedione | 10 mg |
| 5-androstenedione | 10 mg |
| 19-nor-5-androstendione | 10 mg |
| 5-androstenediol | 10 mg |
| 19-nor-5-androstendiol | 10 mg |
| Chrysin | 100 mg |

Two capsules per day is the recommended dosage for an average-weight adult human (70kg).

### EXAMPLE 2

A study of the effect of the deer antler, 4-androstenedione, 19-nor-4-androstenedione, 5-androstenedione, 19-nor-5-androstendione, 5-androstenediol, and 19-nor-5-androstendiol and chrysin in men with age-related decline in testosterone levels, sexual dysfunction and mild depression is conducted over a six-month period. A statistical analysis is performed to compare the resulting testosterone levels of the test and a control (placebo) group to determine if a significant improvement in testosterone levels results from administration of the test preparation. Sixty men having total reduced testosterone and complaining of loss of libido are selected for inclusion in the statistical study. Two weeks prior to the start of the study, each subject completes a self-administered questionnaire to assess sexual function in men with erectile dysfunction. Subjects are asked to rate on a five-point scale the following items: frequency (per week) of morning erections, erectile firmness, ejaculatory frequency (per week) and libido.

Baseline blood samples are drawn on two separate days, measuring free and bound serum testosterone, with standard hemogram and blood chemistry, and the subjects are assigned randomly to one of two treatment groups: the test capsules or matching placebo capsules. Both groups continue on their basal diet and incorporate four capsules of the test composition in the diet.

The effects of the dietary supplementation on total free and bound testosterone, and sexual function as measured by the self-assessment scale are evaluated using multiple linear regression analysis and a standard students T-test. In each analysis, the baseline value of the outcome variable is included in the model as a covariant. Treatment by covariant interaction effects is tested by the method outlined by Weigel and Narvaez 12 Controlled Clinical Trials-378-94 (1991). If there are no significant interaction effects, the interaction terms are removed from the model. The regression model assumptions, normality and homogeneity of variance of residuals, are evaluated by inspecting the plots of residuals versus predicted values. Detection of the temporal outset of effects is done sequentially, by testing for the presence of significant treatment effects at 18, 12, and 6 weeks, proceeding to the earlier time in sequence only when significant effects have been identified at each later time-period. Additionally, differences between groups in nutrient intake, physical activity, and body mass index at each time point are compared using one-way analysis of variance. Changes from the baseline within each group are evaluated using paired T-tests. In additionally, variance analysis is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

A statistically significant increase in testosterone and improved sexual function are observed in the blood of the treated subjects but not the controls upon completion of the study. The differences between the levels of testosterone in the treated subjects and controls are statistically significant.

The invention has been described in detail with particular reference to preferred embodiment thereof. However, it will be appreciated that those skilled in the art, upon consideration of this disclosure may make variations and modifications within the spirit and scope of the invention.

I claim:

1. A composition comprising deer antler; one or more nor-testosterone precursors selected from the croup consisting of 19-nor-4-androstenedione, 19-nor-5-androstenedione, 19-nor-5-androstenediol, and 19-nor-4-androstenediol; and one or more testosterone precursors selected from the group consisting of 4-androstenedione, 5-androstenedione, 5-androstenediol, and 4-androstenediol.

2. The composition of claim 1 further comprising a substance that controls aromatase activity and thus the production of estradiol and estrone.

3. The composition of claim 2 wherein said substance that controls aromatase activity is chrysin.

4. The composition of claim 3 further comprising a substance that controls 5-alpha-reductase activity and thus the production of 5-alpha-diydroxytestosterone.

5. The composition of claim 1 further comprising a substance that controls 5-alpha-reductase activity and thus the production of 5-alpha-diydroxytestosterone.

6. The composition of claim 4 where in said substance that controls 5-alpha-reductase activity and thus the production of 5-alpha-diydroxytestosterone levels is selected from the group consisting of Serenoa repens, cactus flower, zinc, azelaic acid, Dalbergia cochinchinensis, Sabal serrulata, Epilobium, Curcurbitae pepo seeds, Urtica dioica root, and Pollinis siccae extract.

7. The composition of claim 5 wherein said substance that controls 5-alpha-reductase activity and thus the production of 5-alpha-diydroxytestosterone levels is selected from the group consisting of Serenoa repens, cactus flower, zinc, azelaic acid, Dalbergia cochinchinensis, Sabal serrulata, Epilobium, Curcurbitae pepo seeds, Urtica dioica root, and Pollinis siccae extract.

8. The composition of claim 1 wherein said composition is formulated into one or more carriers selected from the group consisting of capsule, tablet, suppository, spray, liquid, powder, liposome, dermal patch, inhalant, and topical cream, lotion or ointment.

9. The composition of claim 1 further comprising a pharmaceutically acceptable carrier.

10. The composition of claim 1 wherein the nor-testosterone precursors and the testosterone precursors are present in equimolar amounts.

11. The composition of claim 1 wherein the deer antler is present in an amount between 1 mg and 2 g, the one or more of the nor-testosterone precursors is present in the composition in an amount between 5 mg and 1 gm, and the one or more of the testosterone precursors is present in an amount between 5 mg and 1 gm.

12. The composition of claim 3 wherein the chrysin is present in amount between 5 mg and 1 gm chrysin.

13. The composition of claim 4 wherein the substance that controls 5-alpha-reductase activity is present in an amount between 5 mg and 1 gm.

14. The composition of claim 11 wherein the deer antler is present in an amount between 5 mg and 500 mg, the one or more of the nor-testosterone precursors is present in the composition in an amount between 5 mg and 500 mg, and the one or more of the testosterone precursors is present in an amount between 5 mg and 500 mg.

15. The composition of claim 12 wherein the chrysin is present in an amount between 5 mg and 500 mg.

16. The composition of claim 13 further wherein the substance that controls 5-alpha-reductase activity is present in an amount between 5 mg and 500 mg.

17. The composition of claim 14 wherein the deer antler is present in an amount between 5 mg and 300 mg, the one or more of the nor-testosterone precursors is present in the composition in an amount between 5 mg and 300 mg, and the one or more of the testosterone precursors is present in an amount between 5 mg and 300 mg.

18. The composition of claim 15 wherein the chrysin is present in an amount between 5 mg and 300 mg.

19. The composition of claim 16 wherein the substance that controls 5-alpha-reductase activity is present in an amount between 5 mg and 300 mg.

20. The composition of claim 17 wherein the deer antler is present in an amount of about 100 mg, the one or more of the nor-testosterone precursors is present in the composition in an amount of about 10 mg, and the one or more of the testosterone precursors is present in an amount of about 10 mg.

21. The composition of claim 18 wherein the chrysin is present in an amount of about 100 mg.

22. The composition of claim 19 wherein substance that controls 5-alpha-reductase activity is present in an amount of about 25 mg.

23. The composition of claim 4 wherein the deer antler, the nor-testosterone precursors, the testosterone precursors, the chrysin, and the substance that controls 5-alpha-reductase are present in amounts sufficient to increase testosterone levels.

24. The composition of claim 4 wherein the deer antler, the nor-testosterone precursors, testosterone precursors, the chrysin, and the substance that controls 5-alpha-reductase activity are present in amounts sufficient to treat sexual dysfunction.

25. The composition of claim 4 wherein the deer antler, the nor-testosterone precursors, testosterone precursors, the chrysin, and the substance that controls 5-alpha-reductase activity are present in amounts sufficient to improve sexual function.

26. The composition of claim 4 wherein the deer antler, the nor-testosterone precursors, testosterone precursors, the chrysin, and the substance that controls 5-alpha-reductase activity are present in amounts sufficient to improve energy.

27. The composition of claim 4 wherein the deer antler, the nor-testosterone precursors, testosterone precursors, the chrysin, and the substance that controls 5-alpha-reductase activity are present in amounts sufficient to enhance feelings of well-being.

28. The composition of claim 4 wherein the deer antler, the nor-testosterone precursors, testosterone precursors, the chrysin, and the substance that controls 5-alpha-reductase activity are present in amounts sufficient to increase muscle mass.

29. A method of increasing testosterone levels in a human male comprising administering to said human a composition comprising deer antler; one or more nor-testosterone precursors selected from the group consisting of 19-nor-4-androstenedione, 19-nor-5-androstenedione, 19-nor-5-androstenediol, and 19-nor-4-androstenediol; and one or more testosterone precursors selected from the group consisting of 4-androstenedione, 5-androstenedione, 5-androstenediol, and 4-androstenediol.

30. The method of claim 29 wherein said composition further comprises a substance that controls aromatase activity and thus the production of estradiol and estrone.

31. The method of claim 30 wherein said substance that controls aromatase activity is chrysin.

32. The method of claim 31 wherein said composition further comprises a substance that controls 5-alpha-reductase activity and thus the production of 5-alpha-diydroxytestosterone.

33. The method of claim 32 wherein said substance that controls 5-alpha-reductase activity and thus the production of 5-alpha-diydroxytestosterone levels is selected from the group consisting of Serenoa repens, cactus flower, zinc, azelaic acid, Dalbergia cochinchinensis, Sabal serrulata, Epilobium, Curcurbitae pepo seeds, Urtica dioica root, and Pollinis siccae extract.

34. The method of claim 29 wherein said composition further comprises a substance that controls 5-alpha-reductase activity and thus the production of 5-alpha-diydroxytestosterone.

35. The method of claim 34 wherein said substance that controls 5-alpha-reductase activity and thus the production of 5-alpha-diydroxytestosterone levels is selected from the group consisting of Serenoa repens, cactus flower, zinc, azelaic acid, Dalbergia cochinchinensis, Sabal serrulata, Epilobium, Curcurbitae pepo seeds, Urtica dioica root, and Pollinis siccae extract.

36. The method of claim 29 wherein said composition is selected from the group consisting of capsule, tablet, suppository, spray, liquid, powder, liposome, dermal patch, inhalant, and topical cream, lotion or ointment.

37. The method of claim 29 wherein said composition farther comprises a pharmaceutically acceptable carrier.

38. The method of claim 29 wherein said composition comprises the nor-testosterone precursors and the testosterone precursors present in equimolar amounts.

39. The method of claim 29 wherein said composition comprises said deer antler in an amount between 1 mg and 2 g, one or more of said nor-testosterone precursors in an amount between 5 mg and 1 gm, and one or more of said testosterone precursors in an amount between 5 mg and 1 gm.

40. The method of claim 31 wherein said composition comprises between 5 mg and 1 gm chrysin.

41. The method of claim 33 wherein said composition comprises between 5 mg and 1 gm of said substance that controls 5-alpha-reductase activity and thus the production of 5-alpha-diydroxytestosterone.

42. The method of claim 35 wherein said composition comprises between 5 mg and 1 gm of said substance that controls 5-alpha-reductase activity and thus the production of 5-alpha-diydroxytestosterone.

43. The method of claim 39 wherein said composition comprises deer antler in an amount between 5 mg and 500 mg, one or more of said nor-testosterone precursors in an amount between 5 mg and 500 mg, and one or more of said testosterone precursors an amount between 5 mg and 500 mg.

44. The method of claim 40 wherein said composition comprises between 5 mg and 500 mg chrysin.

45. The method of claim 41 wherein said composition comprises between 5 mg and 500 mg of said substance that controls 5-alpha-reductase activity and thus the production of 5-alpha-diydroxytestosterone.

46. The method of claim 42 wherein said composition comprises between 5 mg and 500 mg of said substance that controls 5-alpha-reductase activity and thus the production of 5-alpha-diydroxytestosterone.

47. The method of claim 43 wherein said composition comprises deer antler in an amount between 5 mg and 300 mg, one or more of said nor-testosterone precursors in an amount between 5 mg and 300 mg, and one or more of said testosterone precursors in an amount between 5 mg and 300 mg.

48. The method of claim 44 wherein said composition comprises between 5 mg and 300 mg chrysin.

49. The method of claim 45 wherein said composition comprises between 5 mg and 300 mg of said substance that controls 5-alpha-reductase activity and thus the production of 5-alpha-diydroxytestosterone:

50. The method of claim 46 wherein said composition comprises between 5 mg and 300 mg of said substance that controls 5-alpha-reductase activity and thus the production of 5-alpha-diydroxytestosterone.

51. The method of claim 47 wherein said composition comprises said deer antler in an amount of about 100 mg, one or more of said nor-testosterone precursors in an amount of about 10 mg, and one or more of said testosterone precursors in an amount of about 10 mg.

52. The method of claim 48 wherein said composition comprises about 100 mg chrysin.

53. The method of claim 49 wherein said composition comprises about 25 mg of said substance that controls 5-alpha-reductase activity and thus the production of 5-alpha-diydroxytestosterone.

54. The method of claim 50 wherein said composition comprises about 25 mg of said substance that controls 5-alpha-reductase activity and thus the production of 5-alpha-diydroxytestosterone.

55. A method to treat sexual dysfunction in a human male comprising administering to said human a composition comprising deer antler; one or more nor-testosterone precursors selected from the group consisting of 19-nor-4-androstenedione, 19-nor-5-androstenedione, 19-nor-5-androstenediol, and 19-nor-4-androstenediol; and one or more testosterone precursors selected from the group consisting of 4-androstenedione, 5-androstenedione, 5-androstenediol, and 4-androstenediol.

56. The method of claim 55 wherein said composition further comprises a substance that controls aromatase activity and thus the production of estradiol and estrone.

57. The method of claim 56 wherein said substance that controls aromatase activity is chrysin.

58. The method of claim 57 wherein said composition further comprises a substance that controls 5-alpha-reductase activity and thus the production of 5-alpha-diydroxytestosterone.

59. The method of claim 58 wherein said substance that controls 5-alpha-reductase activity and thus the production of 5-alpha-diydroxytestosterone levels is selected from the group consisting of Serenoa repens, cactus flower, zinc, azelaic acid, Dalbergia cochinchinensis, Sabal serrulata, Epilobium, Curcurbitae pepo seeds, Urtica dioica root, and Pollinis siccae extract.

60. The method of claim 55 wherein said composition further comprises a substance that controls 5-alpha-reductase activity and thus the production of 5-alpha-diydroxytestosterone.

61. The method of claim 56 wherein said substance that controls 5-alpha-reductase activity and thus the production of 5-alpha-diydroxytestosterone levels is selected from the group consisting of Serenoa repens, cactus flower, zinc, azelaic acid, Dalbergia cochinchinensis, Sabal serrulata, Epilobium, Curcurbitae pepo seeds, Urtica dioica root, and Pollinis siccae extract.

62. The method of claim 55 wherein said composition is selected from the group consisting of capsule, tablet, suppository, spray, liquid, powder, liposome, dermal patch, inhalant, and topical cream, lotion or ointment.

63. The method of claim 55 wherein said composition further comprises a pharmaceutically acceptable carrier.

64. The method of claim 55 wherein said composition comprises the nor-testosterone precursors and the testosterone precursors present in equimolar amounts.

65. The method of claim 55 wherein said composition comprises said deer antler in an amount between 1 mg and 2 g, one or more of said nor-testosterone precursors in an amount between 5 mg and 1 gm, and one or more of said testosterone precursors in an amount between 5 mg and 1 gm.

66. The method of claim 57 wherein said composition comprises between 5 mg and 1 gm chrysin.

67. The method of claim 59 wherein said composition comprises between 5 mg and 1 gm of said substance that controls 5-alpha-reductase activity and thus the production of 5-alpha-diydroxytestosterone.

68. The method of claim 61 wherein said composition comprises between 5 mg and 1 gm of said substance that controls 5-alpha-reductase activity and thus the production of 5-alpha-diydroytestosterone.

69. The method of claim 65 wherein said composition comprises deer antler in an amount between 5 mg and 500 mg, one or more of said nor-testosterone precursors in an amount between 5 mg and 500 mg, and one or more of said testosterone precursors an amount between 5 mg and 500 mg.

70. The method of claim 66 wherein said composition comprises between 5 mg and 500 mg chrysin.

71. The method of claim 67 wherein said composition comprises between 5 mg and 500 mg of said substance that controls 5-alpha-reductase activity and thus the production of 5-alpha-diydroxytestosterone.

72. The method of claim 68 wherein said composition comprises between 5 mg and 500 mg of said substance that controls 5-alpha-reductase activity and thus the production of 5-alpha-diydroxytestosterone.

73. The method of claim 69 wherein said composition comprises deer antler in an amount between 5 mg and 300 mg, one or more of said nor-testosterone precursors in an amount between 5 mg and 300 mg, and one or more of said testosterone precursors in an amount between 5 mg and 300 mg.

74. The method of claim 70 wherein said composition comprises between 5 mg and 300 mg chrysin.

75. The method of claim 71 wherein said composition comprises between 5 mg and 300 mg of said substance that controls 5-alpha-reductase activity and thus the production of 5-alpha-diydroxytestosterone.

76. The method of claim 72 wherein said composition comprises between 5 mg and 300 mg of said substance that controls 5-alpha-reductase activity and thus the production of 5-alpha-diydroxytestosterone.

77. The method of claim 73 wherein said composition comprises said deer antler in an amount of about 100 mg, one or more of said nor-testosterone precursors in an amount of about 10 mg, and one or more of said testosterone precursors in an amount of about 10 mg.

78. The method of claim 74 wherein said composition comprises about 100 mg chrysin.

79. The method of claim 75 wherein said composition comprises about 25 mg of said substance that controls 5-alpha-reductase activity and thus the production of 5-alpha-diydroxytestosterone.

80. The method of claim 76 wherein said composition comprises about 25 mg of said substance that controls 5-alpha-reductase activity and thus the production of 5-alpha-

[symbol]

81. A method to improve sexual function in a human male comprising administering to said human a composition comprising deer antler; one or more nor-testosterone precursors selected from the group consisting of 19-nor-4-androstenedione, 19-nor-5-androstenedione, 19-nor-5-androstenediol, and 19-nor-4-androstenediol; and one or more testosterone precursors selected from the group consisting of 4-androstenedione, 5-androstenedione, 5-androstenediol, and 4-androstenediol.

82. The method of claim 81 wherein said composition further comprises a substance that controls aromatase activity and thus the production of estradiol and estrone.

83. The method of claim 82 wherein said substance that controls aromatase activity is chrysin.

84. The method of claim 83 wherein said composition further comprises a substance that controls 5-alpha-reductase activity and thus the production of 5-alpha-diydroxytestosterone.

85. The method of claim 84 wherein said substance that controls 5-alpha-reductase activity and thus the production of 5-alpha-diydroxytestosterone levels is selected from the group consisting of Serenoa repens, cactus flower, zinc, azelaic acid, Dalbergia cochinchinensis, Sabal serrulata, Epilobium, Curcurbitae pepo seeds, Urtica dioica root, and Pollinis siccae extract.

86. The method of claim 81 wherein said composition further comprises a substance that controls 5-alpha-reductase activity and thus the production of 5-alpha-diydroxytestosterone.

87. The method of claim 82 wherein said substance that controls 5-alpha-reductase activity and thus the production of 5-alpha-diydroxytestosterone levels is selected from the group consisting of Serenoa repens, cactus flower, zinc, azelaic acid, Dalbergia cochinchinensis, Sabal serrulata, Epilobium, Curcurbitae pepo seeds, Urtica dioica root, and Pollinis siccae extract.

88. The method of claim 81 wherein said composition is selected from the group consisting of capsule, tablet, suppository, spray, liquid, powder, liposome, dermal patch inhalant, and topical cream, lotion or ointment.

89. The method of claim 81 wherein said composition further comprises a pharmaceutically acceptable carrier.

90. The method of claim 81 wherein said composition comprises the nor-testosterone precursors and the testosterone precursors present in equimolar amounts.

91. The method of claim 81 wherein said composition comprises said deer antler in an amount between 1 mg and 2 g, one or more of said nor-testosterone precursors in an amount between 5 mg and 1 gm, and one or more of said testosterone precursors in an amount between 5 mg and 1 gm.

92. The method of claim 83 wherein said composition comprises between 5 mg and 1 gm chrysin.

93. The method of claim 85 wherein said composition comprises between 5 mg and 1 gm of said substance that controls 5-alpha-reductase activity and thus the production of 5-alpha-diydroxytestosterone.

94. The method of claim 87 wherein said composition comprises between 5 mg and 1 gm of said substance that controls 5-alpha-reductase activity and thus the production of 5-alpha-diydroxytestosterone.

95. The method of claim 91 wherein said composition comprises deer antler in an amount between 5 mg and 500 mg, one or more of said nor-testosterone precursors in an amount between 5 mg and 500 mg, and one or more of said testosterone precursors an amount between 5 mg and 500 mg.

96. The method of claim 92 wherein said composition comprises between 5 mg and 500 mg chrysin.

97. The method of claim 93 wherein said composition comprises between 5 mg and 500 mg of said substance that controls 5-alpha-reductase activity and thus the production of 5-alpha-diydroxytestosterone.

98. The method of claim 94 wherein said composition comprises between 5 mg and 500 mg of said substance that controls 5-alpha-reductase activity and thus the production of 5-alpha-diydroxytestosterone.

99. The method of claim 95 wherein said composition comprises deer antler in an amount between 5 mg and 300 mg, one or more of said nor-testosterone precursors in an amount between 5 mg and 300 mg, and one or more of said testosterone precursors in an amount between 5 mg and 300 mg.

100. The method of claim 96 wherein said composition comprises between 5 mg and 300 mg chrysin.

101. The method of claim 97 wherein said composition comprises between 5 mg and 300 mg of said substance that controls 5-alpha-reductase activity and thus the production of 5-alpha-diydroxytestosterone.

102. The method of claim 98 wherein said composition comprises between 5 mg and 300 mg of said substance that controls 5-alpha-reductase activity and thus the production of 5-alpha-diydroxytestosterone.

103. The method of claim 99 wherein said composition comprises said deer antler in an amount of about 100 mg, one or more of said nor-testosterone precursors in an amount of about 10 mg, and one or more of said testosterone precursors in an amount of about 10 mg.

104. The method of claim 100 wherein said composition comprises about 100 mg chrysin.

105. The method of claim 101 wherein said composition comprises about 25 mg of said substance that controls 5-alpha-reductase activity and thus the production of 5-alpha-diydroxytestosterone.

106. The method of claim 102 wherein said composition comprises about 25 mg of said substance that controls 5-alpha-reductase activity and thus the production of 5-alpha-diydroxytestosterone.

107. A method for enhancing feelings of well-being in a human male comprising administering to said human a composition comprising deer antler; one or more nor-testosterone precursors selected from the group consisting of 19-nor-4-androstenedione, 19-nor-5-androstenedione, 19-nor-5-androstenediol, and 19-nor-4-androstenediol; and one or more testosterone precursors selected from the group consisting of 4-androstenedione, 5-androstenedione, 5-androstenediol, and 4-androstenediol.

108. The method of claim 107 wherein said composition further comprises a substance that controls aromatase activity and thus the production of estradiol and estrone.

109. The method of claim 108 wherein said substance that controls aromatase activity is chrysin.

110. The method of claim 109 wherein said composition further comprises a substance that controls 5-alpha-reductase activity and thus the production of 5-alpha-diydroxytestosterone.

111. The method of claim 110 wherein said substance that controls 5-alpha-reductase activity and thus the production of 5-alpha-diydroxytestosterone levels is selected from the group consisting of Serenoa repens, cactus flower, zinc, azelaic acid, Dalbergia cochinchinensis, Sabal serrulata, Epilobium. Curcurbitae pepo seeds, Urtica dioica root, and Pollinis siccae extract.

112. The method of claim 107 wherein said composition further comprises a substance that controls 5-alpha-reductase activity and thus the production of 5-alpha-diydroxytestosterone.

113. The method of claim 112 wherein said substance that controls 5-alpha-reductase activity and thus the production of 5-alpha-diydroxytestosterone levels is selected from the group consisting of Serenoa repens, cactus flower, zinc, azelaic acid, Dalbergia cochinchinensis, Sabal serrulata, Epilobium, Curcurbitae pepo seeds, Urtica dioica root, and Pollinis siccae extract.

114. The method of claim 107 wherein said composition is selected from the group consisting of capsule, tablet, suppository, spray, liquid, powder, liposome, dermal patch, inhalant, and topical cream, lotion or ointment.

115. The method of claim 107 wherein said composition further comprises a pharmaceutically acceptable carrier.

116. The method of claim 107 wherein said composition comprises the nor-testosterone precursors and the testosterone precursors present in equimolar amounts.

117. The method of claim 107 wherein said composition comprises said deer antler in an amount between 1 mg and 2 g, one or more of said nor-testosterone precursors in an amount between 5 mg and 1 gm, and one or more of said testosterone precursors in an amount between 5 mg and 1 gm.

118. The method of claim 109 wherein said composition comprises between 5 mg and 1 gm chrysin.

119. The method of claim 111 wherein said composition comprises between 5 mg and 1 gm of said substance that controls 5-alpha-reductase activity and thus the production of 5-alpha-diydroxytestosterone.

120. The method of claim 111 wherein said composition comprises between 5 mg and 1 gm of said substance that controls 5-alpha-reductase activity and thus the production of 5-alpha-diydroxytestosterone.

121. The method of claim 117 wherein said composition comprises deer antler in an amount between 5 mg and 500 mg, one or more of said nor-testosterone precursors in an amount between 5 mg and 500 mg, and one or more of said testosterone precursors an amount between 5 mg and 500 mg.

122. The method of claim 118 wherein said composition comprises between 5 mg and 500 mg chrysin.

123. The method of claim 119 wherein said composition comprises between 5 mg and 500 mg of said substance that controls 5-alpha-reductase activity and thus the production of 5-alpha-diydroxytestosterone.

124. The method of claim 120 wherein said composition comprises between 5 mg and 500 mg of said substance that controls 5-alpha-reductase activity and thus the production of 5-alpha-diydroxytestosterone.

125. The method of claim 121 wherein said composition comprises deer antler in an amount between 5 mg and 300 mg, one or more of said nor-testosterone precursors in an amount between 5 mg and 300 mg, and one or more of said testosterone precursors in an amount between 5 mg and 300 mg.

126. The method of claim 122 wherein said composition comprises between 5 mg and 300 mg chrysin.

127. The method of claim 123 wherein said composition comprises between 5 mg and 300 mg of said substance that controls 5-alpha-reductase activity and thus the production of 5-alpha-diydroxytestosterone.

128. The method of claim 124 wherein said composition comprises between 5 mg and 300 mg of said substance that controls 5-alpha-reductase activity and thus the production of 5-alpha-diydroxytestosterone.

129. The method of claim 125 wherein said composition comprises said deer antler in an amount of about 100 mg, one or more of said nor-testosterone precursors in an amount of about 10 mg, and one or more of said testosterone precursors in an amount of about 10 mg.

130. The method of claim 126 wherein said composition comprises about 100 mg chrysin.

131. The method of claim 127 wherein said composition comprises about 25 mg of said substance that controls 5-alpha-reductase activity and thus the production of 5-alpha-diydroxytestosterone.

132. The method of claim 128 wherein said composition comprises about 25 mg of said substance that controls 5-alpha-reductase activity and thus the production of 5-alpha-diydroxytestosterone.

133. A method of increasing muscle mass in a human male comprising administering to said human a composition comprising deer antler; one or more nor-testosterone precursors selected from the group consisting of 19-nor-4-androstenedione, 19-nor-5-androstenedione, 19-nor-5-androstenediol, and 19-nor-4-androstenediol; and one or more testosterone precursors selected from the group consisting of 4-androstenedione, 5-androstenedione, 5-androstenediol, and 4-androstenediol.

134. The method of claim 133 wherein said composition further comprises a substance that controls aromatase activity and thus the production of estradiol and estrone.

135. The method of claim 134 wherein said substance that controls aromatase activity is chrysin.

136. The method of claim 135 wherein said composition further comprises a substance that controls 5-alpha-reductase activity and thus the production of 5-alpha-diydroxytestosterone.

137. The method of claim 136 wherein said substance that controls 5-alpha-reductase activity and thus the production of 5-alpha-diydroxytestosterone levels is selected from the group consisting of Serenoa repens, cactus flower, zinc, azelaic acid, Dalbergia cochinchinensis, Sabal serrulata, Epilobium, Curcurbitae pepo seeds, Urtica dioica root, and Pollinis siccae extract.

138. The method of claim 133 wherein said composition further comprises a substance that controls 5-alpha-reductase activity and thus the production of 5-alpha-diydroxytestosterone.

139. The method of claim 138 wherein said substance that controls 5-alpha-reductase activity and thus the production of 5-alpha-diydroxytestosterone levels is selected from the group consisting of Serenoa repens, cactus flower, zinc, azelaic acid, Dalbergia cochinchinensis, Sabal serrulata, Epilobium, Curcurbitae pepo seeds, Urtica dioica root, and Pollinis siccae extract.

140. The method of claim 133 wherein said composition is selected from the group consisting of capsule, tablet, suppository, spray, liquid, powder, liposome, dermal patch, inhalant, and topical cream, lotion or ointment.

141. The method of claim 133 wherein said composition further comprises a pharmaceutically acceptable carrier.

142. The method of claim 133 wherein said composition comprises the nor-testosterone precursors and the testosterone precursors present in equimolar amounts.

143. The method of claim 133 wherein said composition comprises said deer antler in an amount between 1 mg and 2 g, one or more of said nor-testosterone precursors in an amount between 5 mg and 1 gm, and one or more of said testosterone precursors in an amount between 5 mg and 1 gm.

144. The method of claim 135 wherein said composition comprises between 5 mg and 1 gm chrysin.

145. The method of claim 137 wherein said composition comprises between 5 mg and 1 gm of said substance that controls 5-alpha-reductase activity and thus the production of 5-alpha-diydroxytestosterone.

146. The method of claim 139 wherein said composition comprises between 5 mg and 1 gm of said substance that controls 5-alpha-reductase activity and thus the production of 5-alpha-diydroxytestosterone.

147. The method of claim 143 wherein said composition comprises deer antler in an amount between 5 mg and 500 mg, one or more of said nor-testosterone precursors in an amount between 5 mg and 500 mg, and one or more of said testosterone precursors an amount between 5 mg and 500 mg.

148. The method of claim 144 wherein said composition comprises between 5 mg and 500 mg chrysin.

149. The method of claim 145 wherein said composition comprises between 5 mg and 500 mg of said substance that controls 5-alpha-reductase activity and thus the production of 5-alpha-diydroxytestosterone.

150. The method of claim 146 wherein said composition comprises between 5 mg and 500 mg of said substance that controls 5-alpha-reductase activity and thus the production of 5-alpha-diydroxytestosterone.

151. The method of claim 147 wherein said composition comprises deer antler in an amount between 5 mg and 300 mg, one or more of said nor-testosterone precursors In an amount between 5 mg and 300 mg, and one or more of said testosterone precursors in an amount between 5 mg and 300 mg.

152. The method of claim 148 wherein said composition comprises between 5 mg and 300 mg chrysin.

153. The method of claim 149 wherein said composition comprises between 5 mg and 300 mg of said substance that controls 5-alpha-reductase activity and thus the production of 5-alpha-diydroxytestosterone.

154. The method of claim 150 wherein said composition comprises between 5 mg and 300 mg of said substance that controls 5-alpha-reductase activity and thus the production of 5-alpha-diydroxytestosterone.

155. The method of claim 151 wherein said composition comprises said deer antler in an amount of about 100 mg, one or more of said nor-testosterone precursors in an amount of about 10 mg, and one or more of said testosterone precursors in an amount of about 10 mg.

156. The method of claim 152 wherein said composition comprises about 100 mg chrysin.

157. The method of claim 153 wherein said composition comprises about 25 mg of said substance that controls 5-alpha-reductase activity and thus the production of 5-alpha-diydroxytestosterone.

158. The method of claim 154 wherein said composition comprises about 25 mg of said substance that controls 5-alpha-reductase activity and thus the production of 5-alpha-diydroxytestosterone.

65. The method of claim 55 wherein said composition comprises said deer antler in an amount between 1 mg and 2 g, one or more of said nor-testosterone precursors in an amount between 5 mg and 1 gm, and one or more of said testosterone precursors in an amount between 5 mg and 1 gm.

66. The method of claim 57 wherein said composition comprises between 5 mg and 1 gm chrysin.

67. The method of claim 59 wherein said composition comprises between 5 mg and 1 gm of said substance that controls 5-alpha-reductase activity and thus the production of 5-alpha-diydroxytestosterone.

68. The method of claim 61 wherein said composition comprises between 5 mg and 1 gm of said substance that controls 5-alpha-reductase activity and thus the production of 5-alpha-diydroxytestosterone.

69. The method of claim 65 wherein said composition comprises deer antler in an amount between 5 mg and 500 mg, one or more of said nor-testosterone precursors in an amount between 5 mg and 500 mg, and one or more of said testosterone precursors an amount between 5 mg and 500 mg.

70. The method of claim 66 wherein said composition comprises between 5 mg and 500 mg chrysin.

71. The method of claim 67 wherein said composition comprises between 5 mg and 500 mg of said substance that controls 5-alpha-reductase activity and thus the production of 5-alpha-diydroxytestosterone.

72. The method of claim 68 wherein said composition comprises between 5 mg and 500 mg of said substance that controls 5-alpha-reductase activity and thus the production of 5-alpha-diydroxytestosterone.
